(19) United States
(12) Patent Application Publication
(10) Pub. No.: US 2020/0179438 A1
(43) Pub. Date: Jun. 11, 2020

(54) BLOOD GLUCOSE SPIKE SUPPRESSOR, FOOD PRODUCT, AND METHOD FOR PRODUCING BLOOD GLUCOSE SPIKE SUPPRESSOR

(71) Applicant: Takumi SATOH, (US)

(72) Inventors: Takumi SATOH, Tokyo (JP); Hiroto YOKARYO, Okinawa (JP); Morimitsu TERUYA, Okinawa (JP)

(73) Assignee: Takumi SATOH, Tokyo (JP)

(21) Appl. No.: 16/792,891

(22) Filed: Feb. 18, 2020

Related U.S. Application Data
(63) Continuation of application No. PCT/JP2018/030538, filed on Aug. 17, 2018.

(30) Foreign Application Priority Data
Aug. 18, 2017 (JP) 2017-170662

(51) Int. Cl. A61K 31/765; A23L 29/288; A61P 3/08

(52) U.S. Cl. CPC A61K 31/765; A61P 3/08; A23L 29/288

(57) ABSTRACT

A method for producing a blood glucose spike suppressor includes preparing a culture solution containing bacteria capable of accumulating poly(R)-3-hydroxybutyric acid therein, recovering a residue containing granules of poly(R)-3-hydroxybutyric acid by performing a solid-liquid separation on the culture solution, and generating a powder of poly(R)-3-hydroxybutyric acid by drying the residue.

### CROSS-REFERENCE TO RELATED APPLICATIONS

The present application is a continuation application of International Application number PCT/JP2018/030538, filed on Aug. 17, 2018, which claims priority under 35 U.S.C § 119(a) to Japanese Patent Application No. 2017-170662, filed on Aug. 18, 2017. The contents of this application are incorporated herein by reference in their entirety.

### BACKGROUND OF THE INVENTION

### Technical Field

The present invention relates to a blood glucose spike suppressor, a food product, and a method for producing a blood glucose spike suppressor for suppressing spikes in blood glucose level in pets or humans.

Spikes in blood glucose level are considered to be one of the basic factors of lifestyle-related diseases such as obesity, and suppressing spikes in blood glucose level leads to prevention of obesity. Also, it is known that if spikes in blood glucose level can be suppressed, this has significant therapeutic or prophylactic effects on diabetes and diabetes complications characterized by hyperglycemia (see, e.g., Non-Patent Document 1, “One Health Solutions to Obesity in People and Their Pets,” by J. Bartges et al., Journal of Comparative Pathology, Vol. 156, May 2017, pp. 326-333, and Non-Patent Document 2, “β-hydroxybutyrate: Much more than a metabolite,” by John C. Newman et al., Diabetes Research and Clinical Practice, Vol. 106, November 2014, pp. 173-181).

Many dogs and cats tend to get insufficient exercise since they are kept indoors, and there are also many high-calorie pet foods. Therefore, it has been reported that pets are more likely to develop obesity than humans, and more than half of dogs are obese.

As described in Non-Patent Document 1, obese pets are prone to serious chronic diseases (diabetes, dementia, kidney failure, etc.), and this will impose great mental and economic burdens on pet owners. In addition, mental hardship due to the loss of pets is also severe. Pet-related problems, therefore, are no longer just the problem of pet owners but of society as a whole. In particular, the problem of obesity, which causes many lifestyle-related diseases, seems to be rapidly increasing. Therefore, there is a need for supplements that make pets less likely to become obese.

Insulin is known as a hormone that causes obesity. Insulin is released from beta cells of the islets of Langerhans in the pancreas by spiked increases in blood glucose levels. Insulin acts as an “obesity hormone” that causes obesity by promoting the conversion of carbohydrates to fat. Reducing the number of spikes in blood glucose level as much as possible routinely and making the increase of spikes in blood glucose level as gradual as possible leads to the prevention of the development of permanent insulin abnormalities (insulin resistance). In this sense, it is important to suppress spikes in blood glucose level in order to prevent obesity.

In addition, an increasing number of people develop diabetes or exhibit insulin resistance due to lifestyle-related factors such as a decrease in daily exercise. Facing an aging population, diabetics tend to increase explosively year by year, and the increase of diabetics in Japan is serious.

Since there is no radical cure for diabetes, it is important to implement measures to prevent the development of diabetes in humans and pets with insulin resistance. In particular, reducing spikes in blood glucose level to be as small as possible is important for improving hyperglycemia.

Drugs and supplements that can be administered orally are needed to help pets and humans improve their hyperglycemic condition. However, oral agents, especially for pets, that can be administrated orally and can suppress spikes in blood glucose level have not been found yet.

### BRIEF SUMMARY OF THE INVENTION

The present invention focuses on this point and an object of the present invention is to provide a blood glucose spike suppressor which can be administrated orally and can suppress spikes in blood glucose level.

A blood glucose spike suppressor of a first aspect of the present invention is a blood glucose spike suppressor containing a polymer powder of poly(R)-3-hydroxybutyric acid, in which a purity of the poly(R)-3-hydroxybutyric acid is 70% or more and 90% or less, and an average degree of polymerization of the polymer powder is 1900 or more and 2000 or less.

A food product of a second aspect of the present invention is a food product for suppressing spikes in blood glucose level, mixed with a polymer powder of poly(R)-3-hydroxybutyric acid, in which a purity of the poly(R)-3-hydroxybutyric acid is 70% or more and 90% or less, and an average degree of polymerization of the polymer powder is 1900 or more and 2000 or less.

A method for producing a blood glucose spike suppressor of a third aspect of the present invention includes: preparing a culture solution containing bacteria capable of accumulating granules of poly(R)-3-hydroxybutyric acid inside the bacteria; pressurizing the culture solution; recovering a residue containing the granules of poly(R)-3-hydroxybutyric acid by performing a solid-liquid separation on the culture solution after the pressurizing; and generating, by drying the residue, a polymer powder in which (i) a purity of the poly(R)-3-hydroxybutyric acid is 70% or more and 90% or less and (ii) an average degree of polymerization is 1900 or more and 2000 or less.

### BRIEF DESCRIPTION OF THE DRAWINGS

FIG. 1 is a chemical formula showing a structure of poly(R)-3-hydroxybutyric acid (PHB).

FIG. 2 illustrates the principles of suppressing spikes in blood glucose level of PHB which is not powdered.

FIG. 3 shows the principles of an increase of HB purity in the body due to an oral administration of HB.

FIG. 4 shows the principles of the increase of HB purity in the body due to an oral administration of ketone ester (KE: a chemical compound), which is an ester bond of two molecules of HB.

FIG. 5 shows the principles of how spikes in blood glucose level are suppressed by orally administrating powdered PHB.

FIG. 6 is a comparative table of HB, KE, non-powdered PHB, and powdered PHB.

FIG. 7 is a photograph showing the appearance of powdered PHB produced as a blood glucose spike suppressor.

FIG. 8 schematically shows a bacterium in which PHB granules are accumulated.

FIG. 9 is a graph showing changes in blood glucose levels in the body of a person who ingested plain yogurt containing powdered PHB (70% purity).

FIG. 10 is a graph showing changes in blood glucose levels in the body of a person who ingested plain yogurt containing powdered PHB (90% purity).

### DETAILED DESCRIPTION OF THE INVENTION

Hereinafter, the present invention will be described through exemplary embodiments of the present invention, but the following exemplary embodiments do not limit the invention according to the claims, and not all of the combinations of features described in the exemplary embodiments are necessarily essential to the solution means of the invention.

Along with the growing global interest in global environmental issues, there is a growing interest in biodegradable plastics that degrade completely in nature. The inventors of the present application have focused on the fact that some microorganisms have a property of accumulating granules of poly(R)-3-hydroxybutyric acid (hereinafter referred to as PHB), which is used as biodegradable plastic, and the inventors have considered using powdered PHB for the purpose of suppressing spikes in blood glucose level. As described in the present invention, the powdered PHB described in the present specification is clearly different from (i) PHB obtained by chemical synthesis or the like and formed into pellets, (ii) PHB biosynthesized in a bacterial cell while maintaining the crystal structure of PHB, and (iii) PHB biosynthesized in a bacterial cell and formed into granules.

It can be easily inferred that PHB, which is not powdered, has an effect of suppressing spikes in blood glucose level. That is, the non-powdered PHB passes through the small intestine without a physiological effect in the small intestine, is hydrolyzed into HB by intestinal bacteria in the large intestine, is absorbed by animals, and can increase the HB purity of animals. It has been the accepted notion that insulin resistance is improved by persistently increasing HB purity (“Cell Metabolism,” by Pete J. Cox, et al., Aug. 9, 2016, pp. 256-268). Onset of this effect requires time for the intestinal bacteria, which degrade PHB, to grow sufficiently, and if the intestinal bacteria is not sufficiently grown, PHB is excreted with little degradation. That is, there has been a problem that it takes a long time from oral administration of PHB to the onset of its effect, since PHB takes effect by passing through the small intestine and being decomposed and absorbed in the large intestine.

In order to solve this problem, the inventors have confirmed that the magnitude of spikes in blood glucose level can be suppressed by ingesting a food product containing powdered PHB produced from the microorganisms in which the granules of PHB are accumulated, as compared with the case where the same food product containing no powdered PHB is ingested. The inventors have discovered that the powdered PHB exhibits an effect of suppressing spikes in blood glucose level by a principle different from that of non-powdered PHB, and therefore the effect is exhibited in a short time after the oral administration. That is, by powdering PHB, the inventors solved the problem that “it takes at least several days for onset of a physiological effect.”

### [Outline of PHB]

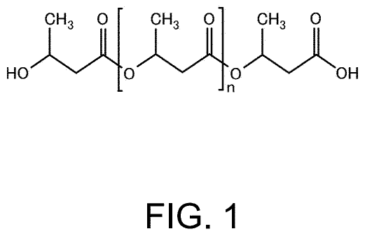

FIG. 1 is a diagram of a chemical formula showing the structure of PHB. PHB is a polymer of (R)-3-hydroxybutyric acid (HB) through an ester bond and is practically insoluble in water. In addition, the ester bond is hydrolysable only by some intestinal bacteria, and mammals do not have an enzyme that hydrolyzes this ester bond. PHB can be produced by fermentation or chemical synthesis methods. PHB has a linear polymer structure formed by the ester bond of HB. When the chemical synthesis method is used, costs for synthesis are high because expensive (R)-3-hydroxybutyric acid is used as a raw material (“Cell Metabolism,” by Pete J. Cox, et al., Aug. 9, 2016, pp. 256-268). On the other hand, in the fermentation method using microorganisms, an inexpensive raw material containing a sugar is used for efficient biosynthesis, and a large amount can be easily produced.

Microorganisms capable of synthesizing PHBs include Halomonas, Bacillus, Azotobactor, Rhizobium, Vibrio, Chromobacterium, Pseudomonas, Micrococcus, Sphaerotailus, Hydrogenomonas, Cupriavidus, Rhodospirillum, Rhodopseudomonas, Chromatium, Spirillum, Comamonas, Aspergillus, Variovorax, Alcaligenes, and Ralstonia. A composition of a culture solution for producing the powdered PHB may be prepared by combining one or more organic carbon sources and one or more nitrogen sources with minerals suitable for each microorganism. Examples of the organic carbon sources include glucose, fructose, mannose, galactose, xylose, arabinose, sucrose, maltose, cellobiose, citric acid, lactic acid, butyric acid, gluconic acid, ethanol, glycerol, and the like. Examples of the nitrogen sources include nitrates (sodium, potassium, calcium, etc.), nitrites, ammonium chloride, ammonium nitrate, ammonium carbonate, ammonium sulfate, urea, etc.

When PHB is produced by the chemical synthesis method, PHB can be produced by chemically coupling (R)-3-hydroxybutyric acid (HB: ketone bodies) using a catalyst. PHB having a weight-average molecular weight of 1,000 or more is desirable in order to (i) lower acidity by reducing carboxylic acid residues and to (ii) have a property of PHB which is different from HB.

### [Suppression of Spikes in Blood Glucose Level]

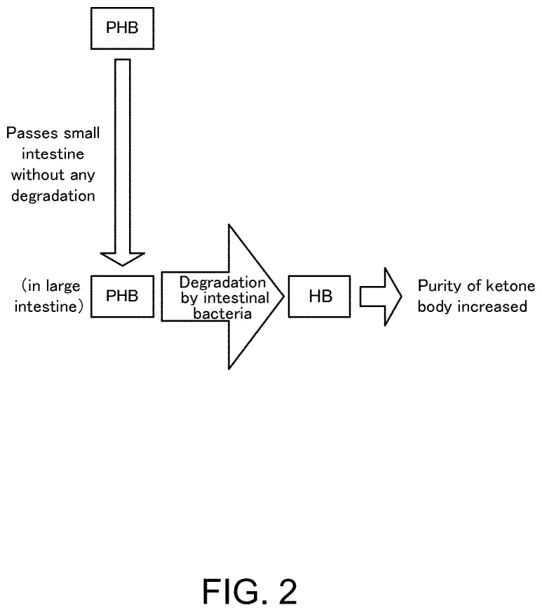

FIG. 2 illustrates the principles of suppressing spikes in blood glucose level of PHB which is not powdered. In the prior art, attention has been paid only to the function of increasing the HB purity in the blood of an animal. Orally administered PHB is delivered to the large intestine without degradation in the stomach or intestine and is degraded by intestinal bacteria in the large intestine. The HB purity is then increased very slowly over a period of several days. It is considered that spikes in blood glucose level are suppressed by increasing the purity of ketone bodies by increasing the HB purity in the body. Therefore, if PHB is not continuously administered for more than a few days, it is considered that intestinal bacteria that degrade PHB are not grown sufficiently, and it requires several days to significantly increase the purity of ketone bodies in the animal.

Here, in order to increase the HB purity, it is conceivable to orally administrate HB itself. In this case, since HB itself is acidic, Na salt of HB or the like is used.

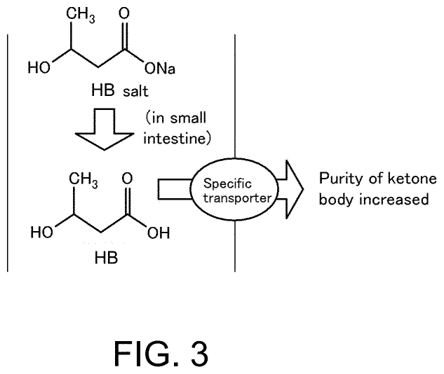

FIG. 3 shows the principles of an increase of the HB purity in the body due to an oral administration of HB. HB salt becomes free acid in an acidic environment of the stomach and is transported into the body by a specific transporter (monocarboxylic acid transporter) in the epithelium of the small intestine, and the HB purity in the body increases several minutes after the ingestion. A few minutes is sufficient for the onset of its effect.

However, Na loading becomes a problem in Na salt of HB. Therefore, it is also conceivable to use ketone ester (KE) (“Cell Metabolism,” by Pete J. Cox, et al., Aug. 9, 2016, pp. 256-268). KE is a compound of HB and 1,3-butanediol through an ester bond.

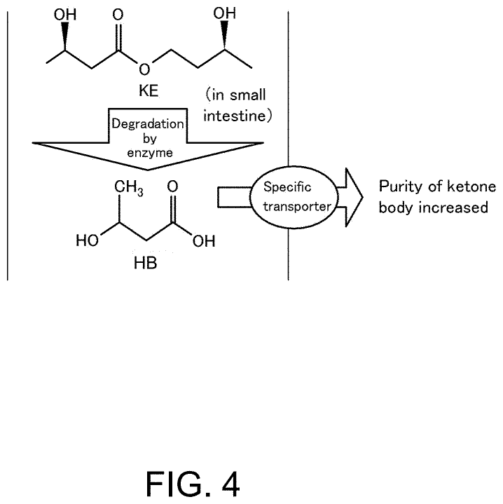

FIG. 4 shows the principles of the increase of the HB purity in the body due to an oral administration of KE. Mammals have an enzyme that hydrolyzes the ester bond of KE, and mammals can rapidly (within minutes) produce high purity of HB. As shown in FIG. 4, KE is degraded by a digestive enzyme in the small intestine to yield two molecules of HB. HB is absorbed by a specific transporter (monocarboxylic acid transporter) in the epithelium of the small intestine, and rapidly increases the HB purity. Therefore, KE has an immediate effect similar to that of HB salt, and although the HB purity in the blood increases to several millimolar (mM) in several minutes, the duration of the onset of the effect is as short as two to three hours (“Cell Metabolism,” by Pete J. Cox, et al., Aug. 9, 2016, pp. 256-268).

Thus, the oral administration of HB or the oral administration of KE can increase HB purity and suppress spikes of blood glucose levels. However, when HB or KE is administered orally, the HB purity decreases within several hours after the increase of the HB purity (“Cell Metabolism,” by Pete J. Cox, et al., Aug. 9, 2016, pp. 256-268).

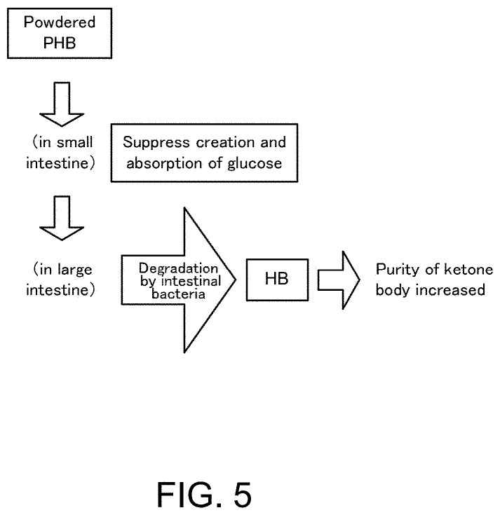

FIG. 5 shows the principles of how spikes in blood glucose level are suppressed by orally administrating the powdered PHB. The powdered PHB causes at least two physiological effects. The first effect is to suppress the degradation or absorption of glucose in the small intestine as PHB polymer. Another effect is that PHB is degraded to HB by an enzyme of intestinal bacteria in the large intestine and absorbed from the epithelium of the large intestine of animals, thereby contributing to an increase in the HB purity. The former effect is caused by powdering PHB. When the powdered PHB is orally administered, the effect of suppressing the absorption of glucose in the small intestine or the conversion of starch (or disaccharide) to glucose occurs in a short time. Therefore, when the powdered PHB is orally administered, it has an immediate effect on the suppression of spikes in blood glucose level. Furthermore, since a process in which the powdered PHB is decomposed by intestinal bacteria in the large intestine to generate HB requires a long time, it is considered that the time in which the HB purity is maintained at high level is longer than that in the case of orally administrating HB or KE. As a result, it is possible to suppress spikes in blood glucose level promptly after the ingestion of the powdered PHB and to continuously maintain the HB purity at a high level, and so it is possible to solve the problems in the case of orally administering HB, KE, and non-powdered PHB.

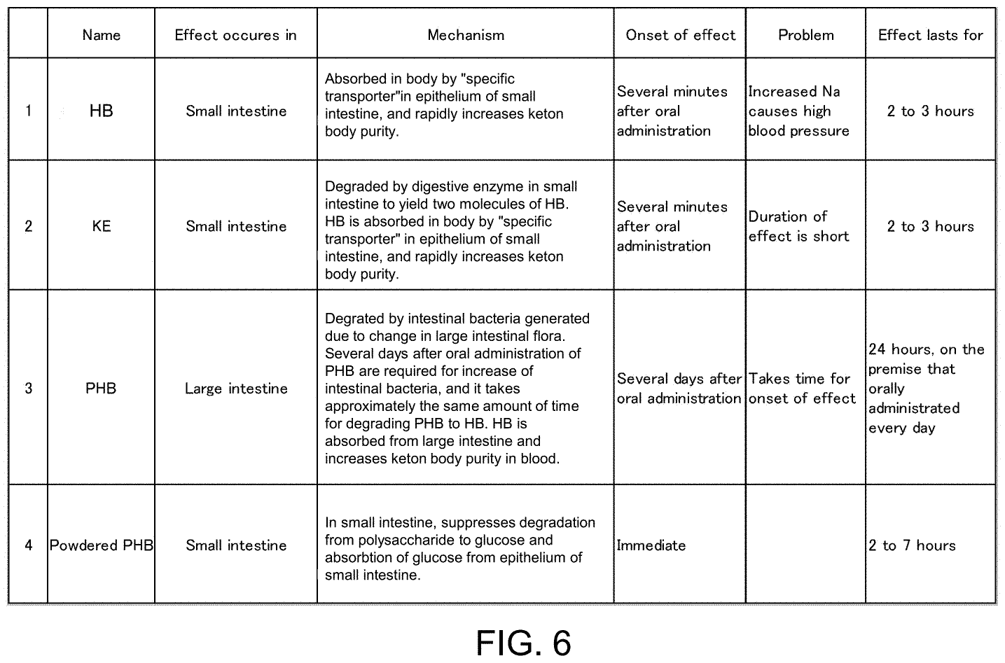

FIG. 6 is a comparative table of HB, KE, non-powdered PHB, and powdered PHB. By orally administering the powdered PHB, as is clear from this comparative table, an effect of suppressing spikes in blood glucose level appears in a short time and the effect lasts for a long period of time, and therefore the powdered PHB is suitable as a blood glucose spike suppressor.

### [Methods for Producing Powdered PHB as the Blood Glucose Spike Suppressor]

PHB can be made as a pharmaceutically acceptable solvate or suspension such as an alcohol suspension (e.g., a methanol suspension, an ethanol suspension) or an ether suspension.

The blood glucose spike suppressor according to the present embodiment can be produced by mixing the powdered PHB, which is an active ingredient, with a physiologically acceptable carrier, excipient, binder, diluent, etc. The blood glucose spike suppressor is produced in forms that can be ingested orally. Orally ingestible forms include food products, granules, powders, tablets (including sugar coated tablets), pills, capsules, syrups, emulsions, suspensions, and the like.

The blood glucose spike suppressor can be formulated with pharmaceutically acceptable carriers such as excipients and additives. The pharmaceutically acceptable excipients and additives include carriers, binders, flavors, buffers, thickeners, colorants, stabilizers, emulsifiers, dispersants, suspending agents, preservatives, and the like. The pharmaceutically acceptable carriers include, for example, magnesium carbonate, magnesium stearate, talc, sugar, lactose, pectin, dextrin, starch, gelatin, tragacanth, methylcellulose, sodium carboxymethylcellulose, low melting wax, cocoa butter, and the like.

Oral agents can be produced by adding excipients (lactose, sucrose, starch, mannitol, etc.), disintegrating agents (calcium carbonate, carboxymethylcellulose calcium, etc.), binding agents (pregelatinized starch, gum arabic, carboxymethylcellulose, polyvinyl pyrrolidone, hydroxypropylcellulose, etc.), lubricating agents (talc, magnesium stearate, polyethylene glycol 6000, etc.), or the like to the active ingredient and compressing the admixture into an appropriate form.

The compression-molded active ingredient is then coated, if necessary, for the purpose of masking the taste, or for the purpose of ensuring the enteric property or durability. Examples of coating agents include ethylcellulose, hydroxymethylcellulose, polyoxyethylene glycol, cellulose acetate phthalate, hydroxypropylmethylcellulose phthalate, and Eudragit (methacrylic acid/acrylic acid copolymer).

The powdered PHB as the blood glucose spike suppressor according to the present embodiment can be incorporated into a food product in the form of a composition or an activating agent containing the powdered PHB. More specifically, the food product according to the present embodiment may be manufactured by mixing the powdered PHB according to the present embodiment, or may be prepared by further admixing various proteins, sugars, fats, trace elements, vitamins, and the like into the manufactured food product; prepared in a liquid state, in a semi-liquid state, or in a solid state; prepared in a paste state; or may be prepared by adding the powdered PHB to food or drink not containing the active ingredient according to the present invention.

In the present invention, the “food product” includes those categorized as health foods, functional foods, foods for specified health use, foods with function claims, or foods for patients. In addition, the term “food product”, when used for a non-human mammal, includes feed and pet foods. The “food product” may be in the form of ordinary food products or in the form of nutritional supplement foods such as supplements.

According to another aspect of the present invention, there is provided a food product containing the powdered PHB as the active ingredient, wherein the food product exhibits a function of treating, preventing, or ameliorating a disease or condition which can be treated, prevented, or ameliorated by increasing the HB purities. According to yet another aspect of the present invention, there is provided a food product containing the powdered PHB as an active ingredient, wherein the food product exhibits an antioxidant ability, an antidote ability, or an anti-inflammatory ability.

In manufacturing drinks provided in the present invention (including drink-type health foods and functional foods), sugars, flavors, juice, food additives, and the like which are used in formulation of ordinary drink products can be appropriately added. Reference may also be made to manufacturing techniques known in the art of manufacturing drinks.

The food product according to the present invention can be made into various forms and can be manufactured in accordance with known technology for manufacturing pharmaceutical products. In such cases, it can be manufactured using the carriers or additives for manufacturing pharmaceutical products as mentioned above in the section for producing blood glucose spike suppressor according to the present invention, more specifically, using the carriers or additives mentioned in the section for oral agents.

When administering or ingesting a composition and food product of the blood glucose spike suppressor, the amount of administration or intake of the powdered PHB according to the present invention can be determined depending on a recipient, recipient's age and body weight, symptoms, the time of administration, the type of dosage form, the method of administration, a combination with other medicines, and the like. For example, when administered as a health food, the powdered PHB according to the present invention can be administered to an adult at a dose ranging from 10 to 2000 mg/kg body weight (preferably 100 to 1000 mg/kg body weight) as the effective amount of powdered PHB, in a single dose or in several divided doses daily. It should be noted that the amount of administration or intake can be calculated and expressed, if necessary, as the amount of PHB for daily administration or intake for an adult having a body weight of 60 kg, assuming that the adult body weight is 60 kg.

### Examples

(Producing the Powdered PHB)

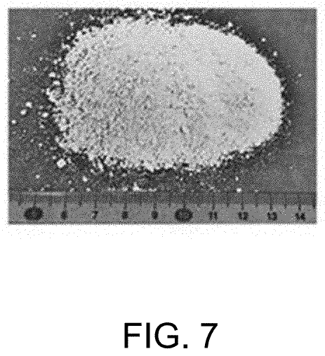

FIG. 7 is a photograph showing the appearance of the powdered PHB produced as the blood glucose spike suppressor. PHB is produced by fermenting with Halomonas sp. OITC1261 (NITE P-02027). The shape of the powdered PHB, in particular the size of particles, can be finely adjusted during a grinding process, which is the final process of the manufacturing.

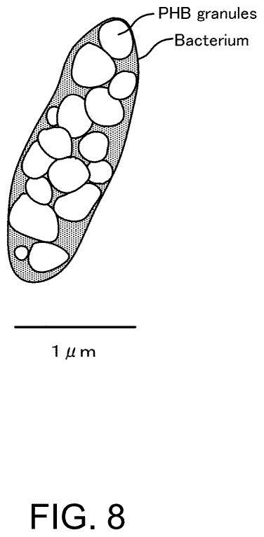

First, a culture solution containing sucrose, as a raw material, and a bacterial cell capable of accumulating PHB inside the cell were prepared. Next, the bacterial cell was cultured in the culture solution. During the culturing, the bacterial cell accumulates PHB within the cell. The culture solution after an aerobic culture contains halomonas bacteria in which PHB granules are accumulated within the bacterial cell, water, and inorganic ions (nitrates, sodium, etc.). OITC1261 produces HB at the same time as PHB, but HB is released outside the bacterial cell (in the culture solution) and is therefore removed later on in a solid-liquid isolation process. Since PHB has a very long chain structure, it is highly folded in the bacterial cell and exists as a very large structure (granular structure of tens to hundreds of nanometers). FIG. 8 schematically shows a bacterium in which PHB granules are accumulated. Depending on culture conditions, PHB granules account for 70% of the volume of the bacterial cell. The composition of the culture solution is, for example, 12.6 g of sodium hydrogen carbonate, 5.3 g of sodium carbonate, 2.0 g of potassium hydrogen phosphate, 1.0 g of salt, 12.5 g of sodium nitrate, 1.0 g of potassium sulfate, 40 mg of magnesium sulfate heptahydrate, 10 mg of calcium chloride dihydrate, 10 mg of ferric sulfate heptahydrate, and 80 mg of disodium edetate with respect to one liter of distilled water. The culture solution may contain 5% w/v of glucose. The culture solution may be added during the culturing of bacteria, as needed. Halomonas may be added, and the aerobic culture may be grown for 3 to 4 days, while being kept at 30° C.

Subsequently, the culture solution containing the bacterial cell was autoclaved. Specifically, after adding 1% to 2% of the surfactant to the culture solution, autoclaving (1.2 atm, 120° C., 20 minutes, 100% humidity) was performed several times. It is presumed that the association state of PHB existing in the granular state due to the association by intermolecular force or hydrogen bonding in the bacterial cell is broken by autoclaving, and thus PHB is formed into powder. As a result, the PHB granules in the bacterial cell can be made into the powdered PHB having a polymerization degree of thousands to tens of thousands. Autoclaving is presumed to break the higher-order structure with which the linear PHB chains are associated, leaving the linear PHB chains unassociated. “Powdered PHB” is a substance in a state where PHB linear chains exist without associating, in contrast to “granules in the bacterial cell where PHB linear chains are associated by weak intermolecular force or hydrogen bonding and form the higher-order structure.” At this time, the degree of polymerization of the powdered PHB is, as estimated from actual measurement values of the molecular weight, several thousands to several tens of thousands.

Subsequently, the culture solution was autoclaved, followed by solid-liquid separation. Specifically, a centrifugal separator was rotated for 5 minutes while a load of 10,000 G was applied to the fermentation solution containing the bacterial cells. Thereafter, a supernatant was discarded to obtain bacterial residue. The obtained residue was washed by repeating a process of adding water, suspending, and then centrifuging three times, thereby separating the residue into a solid and a liquid.

Subsequently, the aqueous solution was discarded, and a residue containing PHB granules, small granules, and bacterial cells (proteins, fats, carbohydrates, moisture) was recovered. The recovered residue was dried at 100° C. for 2 hours and then crushed in a mortar to produce the powdered PHB shown in FIG. 7. Approximately 70% of the components of the produced powdered PHB were PHB. By autoclaving or drying by heat, the cell membrane of the bacterial cell was destroyed, and the PHB granules (number average degree of polymerization of 10,000 or more) in the bacterial cell which formed the higher-order structure by the weak intermolecular force can be converted into powdered PHB having an average degree of polymerization of approximately several thousand.

The purity treatment described above can only be used for food products when bacteria, such as Halomonas, which accumulate large amounts of PHB are used. Bacteria such as Halomonas can accumulate PHB up to 70% of body weight. Therefore, it was confirmed that, by using the bacteria accumulating PHB, PHB can be purified in a range from 70% or more to 90% or less, as described above. In order to purify PHB having a low purity, chloroform or the like is necessary, but it has been confirmed that PHB can be purified in a range from 70% or more to 90% or less, as described above, by using Halomonas bacteria or the like. When the molecular weight distribution of the produced powdered PHB was measured by a GPC method, the weight-average molecular weight was 590,000 and the number average degree of polymerization was 1,939. This numerical value was calculated by 201,671 (number average molecular weight)/104 (molecular weight of HB)=1,939 (number average degree of polymerization).

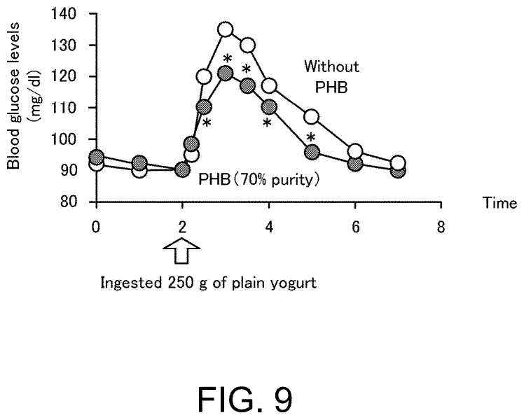

### (Confirmation of Blood Glucose Spike Suppression Effect: FIG. 9)

40 g of the produced powdered PHB (PHB contained therein is 28 g=70% polyketone) was mixed uniformly in plain yogurt (250 g) and consumed by a human. Assuming that an average body weight is 70 kg, the intake of PHB is approximately 400 mg/kg body weight.

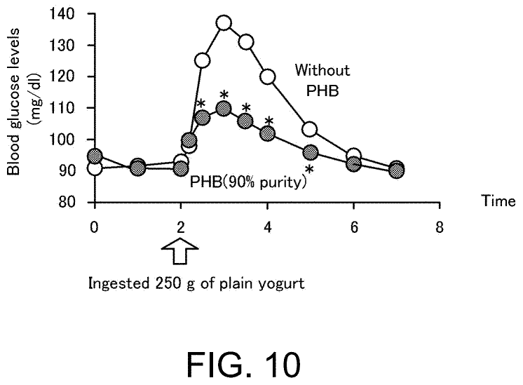

### (Confirmation of Blood Glucose Spike Suppression Effect: FIG. 10)

40 g of the produced powdered PHB (PHB contained therein is 36 g=90% polyketone) was mixed uniformly in plain yogurt (250 g) and consumed by a human. Assuming that an average body weight is 70 kg, the intake of PHB is approximately 514 mg/kg body weight.

Subsequently, blood glucose levels were measured once every other hour using Precision Xceed (Abbott), which uses a blood glucose electrode (FS Precision blood glucose measurement electrode). To ensure that blood glucose levels were stable before consuming PHB and the control (plain yogurt only), a minimum of three measurements were performed, and the measurement results were confirmed to be approximately constant.

FIGS. 9 and 10 are graphs showing changes in blood glucose levels in the body of a person ingesting plain yogurt. The horizontal axis of the graphs shown in FIGS. 9 and 10 indicates time. Blood glucose levels were confirmed to be stable, and then plain yogurt was ingested 2 hours later. The vertical axis indicates blood glucose levels. White circles in FIG. 9 or 10 indicate changes in blood glucose levels when plain yogurt without powdered PHB mixed in is ingested, and black circles indicate changes in blood glucose levels when plain yogurt with powdered PHB (purity of powdered PHB in FIG. 9 is 70% and purity of powdered PHB in FIG. 10 is 90%) mixed in is ingested. As shown in FIGS. 9 and 10, when the plain yogurt mixed with the powdered PHB is ingested, spikes in blood glucose level are reduced in a short time after the ingestion. Further, it was at least confirmed that the blood glucose levels were suppressed to be lower for a long time when the plain yogurt mixed with the powdered PHB was ingested than when the plain yogurt not mixed with the powdered PHB was ingested. When a person eats yogurt, sugars contained in the yogurt are rapidly decomposed, and the blood glucose levels increase rapidly. This rapid increase in blood glucose levels induces an insulin spike. However, when 40 g of powdered PHB with plain yogurt was ingested, spikes in blood glucose level were suppressed significantly.

### [Effects of the Blood Glucose Spike Suppressor of the Present Embodiment]

By mixing the powdered PHB, which is the blood glucose spike suppressor according to the present embodiment, with drinks, it is possible to suppress spikes in blood glucose level in a short time and maintain the effect of suppressing spikes in blood glucose level for a long time. Since the powdered PHB is tasteless and odorless, it is easy to mix the powdered PHB with pet foods, and therefore it is possible to have pets routinely consume the powdered PHB every day. The effect of suppressing spikes in blood glucose level, which is supposed to be the physiological effect in the small intestine, is obtained by using the powdered PHB, and is a novelty presented in the present invention for the first time. As shown in FIGS. 9 and 10, it was confirmed that the powdered PHB having a purity of 70% and the powdered PHB having a purity of 90% significantly suppressed spikes in blood glucose level. In FIG. 10, the suppression of the spikes in blood glucose level is significantly stronger than that in FIG. 9, and therefore, it is reasonable to infer that the powdered PHB having a purity of 90% or more can suppress spikes in blood glucose level more strongly. That is, for example, if the purity is purified to a purity of 99% or more, it can be easily inferred that it becomes a stronger blood glucose spike suppressor. In addition, from the results shown in FIGS. 9 and 10, it is considered that the effect occurs when the powdered PHB has a purity of 60% or more.

Also, the powdered PHB can be produced in large quantities using microorganisms and manufactured at low cost because inexpensive raw materials such as molasses can be used.

The present invention is explained on the basis of the exemplary embodiments. The technical scope of the present invention is not limited to the scope explained in the above embodiments and it is possible to make various changes and modifications within the scope of the invention. For example, the specific embodiments of the distribution and integration of the apparatus are not limited to the above embodiments, all or part thereof, can be configured with any unit which is functionally or physically dispersed or integrated. Further, new exemplary embodiments generated by arbitrary combinations of them are included in the exemplary embodiments of the present invention. The effect of the new embodiment caused by the combination has the effect of the original embodiment together.

What is claimed is:

1. A blood glucose spike suppressor containing a polymer powder of poly(R)-3-hydroxybutyric acid, in which a purity of the poly(R)-3-hydroxybutyric acid is 70% or more and 90% or less, and an average degree of polymerization of the polymer powder is 1900 or more and 2000 or less.

2. A food product for suppressing spikes in blood glucose level, mixed with a polymer powder of poly(R)-3-hydroxybutyric acid, in which a purity of the poly(R)-3-hydroxybutyric acid is 70% or more and 90% or less, and an average degree of polymerization of the polymer powder is 1900 or more and 2000 or less.

3. A method for producing a blood glucose spike suppressor, comprising:
preparing a culture solution containing bacteria capable of accumulating granules of poly(R)-3-hydroxybutyric acid inside the bacteria;
pressurizing the culture solution;
recovering a residue containing the granules of poly(R)-3-hydroxybutyric acid by performing a solid-liquid separation on the culture solution after the pressurizing; and
generating, by drying the residue, a polymer powder in which (i) a purity of the poly(R)-3-hydroxybutyric acid is 70% or more and 90% or less and (ii) an average degree of polymerization is 1900 or more and 2000 or less.
